(19) United States
(12) Patent Application Publication
(10) Pub. No.: US 2012/0329092 A1
(43) Pub. Date: Dec. 27, 2012

(54) PROCESS FOR THE PURIFICATION OF GLYCOPROTEINS

(75) Inventors: Steffen Goletz, Berlin (DE); Lars Stöckl, Berlin (DE)

(73) Assignee: Glycotope GmbH, Berlin (DE)

(21) Appl. No.: 13/575,068

(22) PCT Filed: Jan. 25, 2011
(86) PCT No.: PCT/EP2011/000302

§ 371 Date: Sep. 6, 2012

(30) Foreign Application Priority Data
Jan. 28, 2010 (EP) 10 000 867.1

(51) Int. Cl. C07K 1/36; C12P 21/00

(52) U.S. Cl. 435/69.4; 530/395; 530/380; 435/69.1

(57) ABSTRACT

The present invention relates to a process for the purification of a glycoprotein comprising subjecting a liquid containing said glycoprotein to the steps of:
a) reverse phase chromatography,
b) boronate affinity chromatography, and
c) size exclusion chromatography.

Also provided is a manufacturing process for producing a glycoprotein of interest.

The present invention relates to a process for the purification of glycoproteins, such as in particular EPO (erythropoietin), and to the manufacturing of a recombinant glycoprotein of interest employing a respective purification process.

Glycoproteins are proteins that contain oligosaccharide chains covalently attached to polypeptide side-chains. Glycoproteins can have a vast number of different biological functions including structural, protective, carrier, hormone or enzyme functions. Accordingly various glycoproteins can be used as pharmaceuticals. The provision of such glycoproteins is thus highly desirable. Several glycoproteins can nowadays be produced recombinantly, which however requires extensive purification procedures to extract the targeted glycoprotein from the cell culture harvest.

Cytokines represent an important class of glycoproteins. They have regulatory functions on the proliferation and differentiation of target cells. Cytokines can be classified into the groups of interferones, interleukins, colony stimulating factors (CSF), tumor necrosis factors (TNFs) and chemokines.

An important cytokine is erythropoietin. Erythropoietin is a glycoprotein hormone that is a cytokine for erythrocyte precursors in the bone marrow. As such, it controls red blood cell production (erythropoiesis) and also has other known biological functions. For example, erythropoietin plays an important role in the brain's response to neuronal injury and is also involved in the wound healing process.

Human EPO is expressed as prepro-protein having 193 amino acids and is then posttranslationally processed into a 165 amino acid glycoprotein bearing one O-glycosidic carbohydrate structure at Ser 126 and three N-glycosidic carbohydrate structures at Asn 24, Asn 38 and Asn 83. The natural EPO normally is sialylated, having sialic acid residues at the end of its carbohydrate chains. The presence of this sialic acid residues increase the circulation half-life of EPO in human bodies.

In nature, erythropoietin is produced by the peritubular capillary endothelial cells in the kidney. Furthermore, it is available as a therapeutic agent produced by recombinant DNA technology in mammalian cell culture. It is used in treating anaemia resulting from chronic kidney disease and myelodysplasia, from the treatment of cancer (chemotherapy and radiation), and from other critical illnesses such as heart failure.

In addition to its well-known role in erythropoiesis, EPO also plays an important protective role in the nervous system. However, the administration of large amounts of EPO leads to potentially harmful increases in red cell mass and to the production of hyperreactive platelets, which can lead to thrombosis. While effective production of red blood cells needs continuous presence of EPO, a brief exposition is sufficient for neuroprotection. Asialo-erythropoietin (asialo-EPO)—a desialylated form of EPO—is rapidly cleared from the blood and does not increase erythrocyte mass, but still shows protective activity in animal models for stroke, spinal cord injury, and peripheral neuropathy.

Because of the importance of EPO in the treatment of anaemia and neurological diseases, the provision of EPO of high purity and high specific activity is desirable. EPO treatment requires repeated administrations. Highly purified EPO preparations can reduce the amount of medicament the patient has to take and allows a much more precise determination of the amount of EPO used for medication.

There are various purification methods known in the art for isolating EPO from natural or recombinant sources. However, these methods suffer from a large number of different purification steps, in particular multiple chromatography steps, and the need for buffer exchange and concentration steps in between these chromatography steps. Furthermore, purity and yield of these methods often are not optimal.

The object of the present invention therefore is to provide a purification process which renders glycoproteins such as EPO in high yield and purity, using a minimum of different purification steps.

Accordingly the present invention relates to a purification process for glycoproteins such as EPO comprising subjecting a liquid containing the glycoprotein to the following steps:
a) reverse phase chromatography (RPC);
b) boronate affinity chromatography (BAC); and
c) size exclusion chromatography (SEC).

The steps (a), (b), and (c) may be carried out in any order. It is preferred that reverse phase chromatography or boronate affinity chromatography is performed as the first of the three chromatography steps. In more preferred embodiment reverse phase chromatography is performed as the first of the three chromatography steps.

The purification process may optionally comprise additional steps, e.g. ion exchange chromatography such as anion exchange chromatography or cation exchange chromatography, chromatofocusing, affinity chromatography such as dye affinity chromatography, immune affinity chromatography, lectin affinity chromatography or hydrophobic interaction chromatography, filtration such as diafiltration, ultrafiltration or nanofiltration, or at least one virus inactivation step. In a preferred embodiment the process of the present invention includes an anion exchange chromatography (AEX) as a fourth chromatography step.

In a preferred embodiment the steps (a), (b) and (c) are performed in the sequence of
(1) reverse phase chromatography,
(2) boronate affinity chromatography, and
(3) size exclusion chromatography.

Performing RPC as first chromatography step is preferred because this embodiment provides the option to load rather “raw” biological liquids such as crude glycoprotein, natural source liquids, cell culture medium or cell lysates directly onto the RPC, optionally after a clearing (e.g. filtration), concentration and/or buffer exchange step as described below. This embodiment provides the advantage that even when using such sample liquids high amounts of the sample can be loaded onto the chromatography column without the danger of clogging or overloading the column. Furthermore, the buffer conditions required for RPC do not lead to excessive aggregation of components of the sample solution. In summary, using RPC as first chromatography step reduces the number of preparation steps which are necessary before starting the chromatographic purification and allows the use of high amounts of sample solution with high amounts of other components besides the glycoprotein of interest.

In another preferred embodiment, the process according to the invention further comprises an anion exchange chromatography step (d). Preferably, the anion exchange chromatography is performed subsequent to size exclusion chromatography (c). As described above, additional steps may be performed in addition to and also between the steps. However, one important finding of the inventors is that surprisingly binding of the glycoprotein to the boronate affinity material in step (b) can be achieved in the presence of an organic solvent which may preferably be used in the elution step of the reverse phase chromatography. Therefore, preferably no buffer exchange steps such as diafiltration and/or concentration steps such as ultrafiltration are performed between the reverse phase chromatography and the boronate affinity chromatography. Furthermore, it has also been found that the glycoprotein eluted in the boronate affinity chromatography can directly be used in the subsequent size exclusion chromatography. Therefore, preferably no buffer exchange steps such as diafiltration and/or concentration steps such as ultrafiltration are performed between the boronate affinity chromatography and the size exclusion chromatography.

The purification method of the invention provides the glycoprotein such as EPO in high purity, which may then be formulated as a pharmaceutical composition. The purity in general is above 90%, preferably >95% w/w, more preferably >99% w/w, even more preferably >99.5% w/w, based on total protein.

The crude glycoprotein which forms the starting material for the purification process according to the present invention may be provided in liquids of natural sources or by recombinant techniques such as e.g. in cell culture harvests containing the glycoprotein. Typically, the starting material as obtained from a natural source or a cell harvest, preferably from a cell harvest, is clarified first (e.g. by filtration) and then optionally concentrated (e.g. by using ultrafiltration) and/or buffer exchanged (e.g. through a diafiltration step) prior to being captured by the first chromatographic step.

In the steps of chromatography typically commercially available resins are used, preferably polymer-based resins or agarose-based resins. It is also possible to use membrane chromatography in which the resin is replaced by a functionalised membrane such as Sartobind™ membranes (Sartorius) or ChromaSorb™ (Millipore).

The steps of the purification process of the present invention are outlined in the following in more detail.

Reverse Phase Chromatography Step (a)

The process involves a step of reverse phase chromatography (a). In a preferred embodiment, especially in the case of recombinant glycoproteins, the reverse phase chromatography is used as capture step in which the glycoprotein is enriched from the natural source liquid or the cell culture harvest. It is preferred to perform a virus inactivation subsequent to elution from the RPC column.

“Reverse phase chromatography” according to the invention in particular refers to a chromatography step wherein a non-polar stationary phase and preferably a polar mobile phase are used. In reverse phase chromatography, normally polar compounds are eluted first while non-polar compounds are retained.

The reverse phase chromatography is usually performed by equilibrating and loading the column, followed by a wash and subsequent elution, each with a buffer preferably containing an organic solvent such as acetonitrile or isopropanol. The organic solvent such as isopropanol can be used for virus inactivation subsequent to elution.

The equilibration, load, wash and elution are preferably carried out by using a mobile phase at acidic conditions, for example at a pH of 4 or less, preferably at a pH in the range from 3 to 1, more preferably at about pH 2. A preferred acid for adjusting the pH of the mobile phase is trifluoroacetic acid (TFA), which preferably is used in an amount of from 0.01 to 1% (v/v), more preferably about 0.1% (v/v). However, also other acids can be used, in particular organic acids such as trichloroacetic acid, maleic acid and oxalic acid, or inorganic acids such as hydrochloric acid, sulfuric acid and phosphoric acid.

However, in another embodiment the equilibration, load, wash and elution can also be carried out by using a mobile phase buffering at mildly alkaline pH, for example at or about pH 7 to 8.5, more preferably at or about 7.5. In a preferred embodiment, the buffering species is a phosphate buffer, preferably sodium phosphate. Alternate buffers adequate for a pH at or around 7.5 include BES, MOPS, ammonium acetate, TES, HEPES.

It is preferred that no buffer exchange is performed after step (a) in case that subsequently step (b) (BAC) is performed. In particular, it has been found that binding to the boronate affinity material also occurs in the presence of the organic solvent used for elution during the RPC. Thus, buffer exchange steps between the RPC and the BAC can be omitted, saving time, reducing complexity of the purification process and reducing protein loss.

In a preferred embodiment the buffer solutions used for the RPC step contain an organic solvent, the concentration of which is modulated for different phases of the chromatography step (equilibration, load, wash and elution). Preferably the organic solvent is a water miscible organic solvent such as acetonitrile or an alcohol (such as methanol, ethanol, etc.), more preferably isopropanol.

In the equilibrating and loading buffer solution and in the wash buffer solution the organic solvent is preferably contained in an amount between 5 and 15% v/v of total buffer solution, preferably between 5 and 12% v/v of total buffer solution. The wash buffer is typically identical to the loading buffer. In the elution buffer solution the organic solvent is preferably contained in a higher amount than in the loading buffer. In a preferred embodiment, elution is performed by gradually increasing the concentration of the organic solvent in the mobile phase. In particular, a linear gradient can be used starting at the organic solvent concentration of the wash buffer and ending at a much higher concentration, for example at 25% (v/v) or more, preferably at 40% (v/v) or more, 50% (v/v) or more, 60% (v/v) or more, or 80% (v/v) or more.

In preferred embodiments, the reverse phase chromatography step can include a virus inactivation step. Virus inactivation may be achieved by incubating the protein loaded onto, bound to or eluted from the column in the presence of an organic solvent, preferably isopropanol, acetonitrile or ethanol. The incubation time and incubation temperature preferably are chosen so as to effect a desired degree of virus inactivation and in particular depend on the concentration and nature of the organic solvent used. Furthermore, these parameters should also be adjusted depending on the stability of the glycoprotein to be purified. For example, the protein is incubated for at least 15 min, preferably for at least 30 min, at least 45 min, at least 1 h, at least 2 h, at least 3 h or at least 6 h. The incubation can be performed at low temperature such as at or below 4° C. or at or below 10° C., or it can be performed at about room temperature. The incubation can be performed directly after the sample has been loaded onto the column, during or after the washing step, after applying the elution buffer but prior to elution of the glycoprotein, or after elution of the glycoprotein. If isopropanol is used as the organic solvent, virus inactivation is preferably done at an isopropanol concentration of at least 15% (v/v). In this case, the glycoprotein is preferably incubated for about 2 h, preferably at room temperature. Preferably, the virus inactivation is performed after elution of the glycoprotein from the reverse phase chromatography column, preferably in the elution buffer used. However, optionally further components may be added to the glycoprotein solution after elution from the column, in particular for enhancing the virus inactivation and/or the glycoprotein stability.

A similar effect can be obtained by performing a virus inactivation step during the RPC using incubation at low pH. Incubation of the glycoprotein sample at low pH such as a pH at or below about 4, preferably a pH between 1 and 3, more preferably a pH at about 2, effectively inactivates viruses. For adjusting the pH of the solution, acids as listed above can be used. In particular, incubation is preferably performed in the loading buffer, the washing buffer, the elution buffer or a mixture thereof, preferably in the buffer in which the glycoprotein to be purified is eluted from the reverse phase column. Again, the incubation conditions such as incubation time, incubation temperature and solution components preferably are chosen so as to effect a desired degree of virus inactivation and also should be adjusted depending on the stability of the glycoprotein to be purified at the chosen pH value. The incubation conditions are preferably chosen as discussed above for the virus inactivation step via incubation in the presence of an organic solvent. In preferred embodiments, the RPC comprises a virus inactivation step wherein the glycoprotein sample is incubated in the presence of an organic solvent as described above and at the same time at a low pH value as described above, combining both virus inactivation effects.

Using a virus inactivation step during the RPC, the process of the invention may be performed without any further virus inactivation step. However, various virus inactivation steps may also be combined, for example a virus inactivation during RPC and a virus inactivation via nanofiltration and/or via pH adjustment as described herein.

In a particularly preferred embodiment, the product-contacting buffers for the step of RPC (equilibration, load, wash, elution) contain an antioxidant, such as L-methionine. Alternate antioxidants include t-butyl-4-methoxyphenol, 2,6-bis(1,1-dimethylethyl)-4-methyl phenol; potassium or sodium bimetabisulfite, sodium bisulfite.

Reversed phase column material is made of a resin to which a hydrophobic material may be attached. Typical column materials are silica and polystyrene; hydrophobic ligands may optionally be attached. In case of substituted resins, the resin is substituted with a hydrophobic ligand, typically selected from (but not limited to) aliphates, such as C₂, C₄, C₆, C₈, C₁₀, C₁₂, C₁₄, C₁₆, or C₁₈ or derivates of these, e.g. cyanopropyl (CN-propyl), or branched aliphates, or benzene-based aromates, such as phenyl, or other polar or non-polar ligands. The ligand may be a mixture of two or more of these ligands. Suitable polystyrene based resins include, without limitation, resins supplied by Rohm Haas (e.g. Amberlite XAD or Amberchrom CG), Polymer Labs (e.g. PLRP-S), GE Healthcare (e.g. Source RPC), Applied Biosystems (e.g. Poros R). A particularly preferred resin is Source 30 RPC (GE Healthcare).

The manufacturing processes for and optimal features of the column material often require that a linking group also called a spacer is inserted between the resin and the ligand. Other parameters in the methods of the present invention include load, i.e. amount of protein which is loaded to the column and flow rate. These parameters may be optimised through experiments which are known to the person skilled in the art.

The glycoprotein is typically loaded onto the column in a concentration of at least about 0.1 mg per ml of resin, such as, e.g., at least about 0.2 mg, 0.5 mg, 1 mg, 2 mg, 5 mg, 10, or 20 mg per ml of resin; or in the range of 0.1-200 mg, such as, e.g., 0.1-100 mg, 0.5-100 mg, 1-50 mg, or 2-30 mg per mL of resin; preferably the load is at least 1 mg per mL resin. Measurement of packed resin volume is typically done in suspension or similar mode.

Boronate Affinity Chromatography Step (b)

The purification process according to the invention further comprises a boronate affinity chromatography step. Herein, in particular glycoproteins are enriched as they are capable to bind to the boronate affinity material. This step preferably is used as second chromatography step after an initial reverse phase chromatography.

“Boronate affinity chromatography” according to the invention in particular refers to chromatography steps wherein a boronate affinity material is used. Boronate affinity materials preferably comprise as binding ligands boronic acid groups, i.e. dihydroxyboronyl groups, preferably attached to a hydrocarbon. Preferably, the boronic acid is attached to an aromatic carbon atom such as to a phenyl group. Particularly preferred for use in the process of the invention are boronate affinity materials bearing phenylboronate groups. The boronate affinity material is capable of (selectively) binding to and interacting with 1,2-cisdiol structures on organic compounds. Since the carbohydrate chains of glycoproteins comprise such structures, boronate affinity material can be used for separating glycoproteins from other compounds not comprising a 1,2-cisdiol structure or a similar structure.

The boronate affinity chromatography comprises an optional equilibration step, a loading step, an optional washing step and an elution step. In the equilibration step, the boronate affinity column is equilibrated with a suitable equilibration buffer which preferably is identical to the loading buffer and/or the washing buffer. Equilibration is preferably performed until substantially the entire mobile phase in the column is exchanged with the equilibration buffer, for example using at least 1 CV (column volume) equilibration buffer, more preferably at least 2 CV, 3 CV, 4 CV or at least 5 CV.

In the loading step, the material containing the glycoprotein of interest is loaded onto the boronate affinity column. In preferred embodiments, boric acid buffer is added to the glycoprotein solution prior to loading of the boronic affinity column. The boric acid in the glycoprotein solution is used for reducing unspecific binding to the boronic affinity material. The boric acid buffer preferably contains 0.01 to 1 M boric acid, more preferably 0.05 to 0.5 M and most preferably about 0.1 to about 0.2 M boric acid, and preferably has a pH in the range of 7 to 9, preferably about 8.5. The boric acid buffer preferably is added to the glycoprotein solution in a ratio of about 1:2 to about 20:1 (volume(boric acid buffer):volume(glycoprotein solution)), preferably about 2:1 to about 10:1, more preferably about 5:1. The addition of the boric acid buffer is particularly preferred when the boronic affinity chromatography is performed directly after the reverse phase chromatography.

The boronate affinity chromatography preferably comprises a washing step wherein unbound and/or only weakly bound components of the sample loaded onto the column are washed away and thereby separated from the glycoprotein bound to the column. The washing step is performed by washing the column with at least 1 CV, preferably at least 1.5 CV, 2 CV, 2.5 CV or at least 3 CV of washing buffer.

The equilibration buffer, loading buffer and washing buffer may be the same or different and preferably are identical in composition. Examples of suitable equilibration buffers, loading buffers and washing buffers are boric acid, phosphate, glycine, MES, Bis-Tris, ADA, PIPES, ACES, BES, MOPS, TES, HEPES. Boric acid buffers preferably comprise 0.01 M to 1 M, preferably 0.05 to 0.2 M, more preferably about 0.1 M boric acid, and optionally a salt such as NaCl, in particular about 0.01 M to 1 M NaCl, preferably 0.1 M to 0.5 M NaCl, for example about 0.15 M NaCl, preferably having a pH of about 7 to about 9, preferably about 8.5. The phosphate buffer preferably is phosphate buffered saline having a pH of about 7 to about 9, for example about 7.4 or about 8.3. The glycine buffer preferably comprises about 0.1 to about 3 M glycine, preferably about 0.5 to about 1.5 M, more preferably about 1 M glycine, and optionally additionally comprises a salt such as NaCl, in particular about 0.01 M to 1 M NaCl, preferably 0.1 M to 0.5 M NaCl, for example about 0.15 M NaCl, preferably having a pH of about 7 to about 9, preferably about 8.6. A boric acid buffer comprising about 0.1 M boric acid and about 0.15 M NaCl at a pH of about 8.5 is preferred as equilibration, loading and washing buffer.

In the elution step of the boronate affinity chromatography, the bound glycoprotein is eluted with an elution buffer which is capable of disrupting the interaction of the glycoprotein and the boronate affinity material. In particular, elution buffers comprising a compound having a 1,2-cisdiol structure or a similar structure such as a 1,2-diol structure or a 1,3-diol structure are used. Examples of suitable elution buffers are citrate buffers and tris buffers. The tris buffer preferably comprises 0.05 M to 1 M tris, preferably 0.1 to 0.5 M tris, more preferably about 0.2 M tris, preferably having a pH in the range of 2 to 10, preferably about 7 to about 9, more preferably about 7.5 to about 8.0. The citrate buffer preferably comprises 0.02 M to 1 M citrate, preferably 0.05 to 0.2 M citrate, more preferably about 0.1 M citrate, preferably having a pH in the range of 2 to 10, preferably about 4 to about 7, more preferably about 5.0 to about 6.0. The elution buffer may optionally comprise salt such as NaCl, for example about 1 mM to about 1 M NaCl, preferably about 10 mM NaCl. Elution preferably is performed in a stepwise manner, exchanging the loading or washing buffer by the elution buffer. However, elution may also performed by changing the buffer gradually. Preferably, the fractions comprising the glycoprotein of interest are collected and pooled.

In preferred embodiments, the glycoprotein of interest is kept in its natural state, in particular in its natural three-dimensional structure or its natural fold during elution. Elution is preferably performed under non-denaturing conditions with respect to the glycoprotein. In particular, denaturing conditions are avoided during elution or during the entire boronate affinity chromatography step or during the entire purification process according to the invention. In particular, elution of the glycoprotein is performed without using urea.

As boronate affinity material, a chromatography material comprising boronic acid groups is used. Preferably, the material comprises phenyl boronic acid groups, in particular m-amiophenyl boronic acid groups. Suitable material are for example phenyl boronate resins such as phenyl boronate agarose, or beads such as controlled pore glass beads or polymeric beads functionalized with phenyl boronic acid ligands such as m-aminophenyl boronic acid. Particular examples are ProSep-PB from Millipore Corporation, Billerica, Mass., USA.

In a particularly preferred embodiment, the product-contacting buffers for the step (b) of BAC (equilibration, load, wash, elution) contain an antioxidant, such as L-methionine. Alternative antioxidants include t-butyl-4-methoxyphenol, 2,6-bis(1,1-dimethylethyl)-4-methyl phenol; potassium or sodium bimetabisulfite, sodium bisulfite.

### Size Exclusion Chromatography Step (c)

The process of the present invention also involves a step of size exclusion chromatography (c), e.g. for further purifying and/or re-buffering of the glycoprotein. The size exclusion chromatography comprises the step of equilibrating and loading the eluate of the previous chromatography step to a gel filtration matrix equilibrated with a buffer having a composition which is desired for storage or further processing of the glycoprotein at a pH of typically between 6.5 and 9, preferably about 7.5.

For performing size exclusion chromatography, the gel is typically selected from the groups of polymeric gels including, but not limited to dextran-based gels such as Sephadex (e.g. Sephadex G-25) or polyacrylamide gels such as Sephacryl (e.g. Sephacryl-5400), agarose-based gels such as Superose or Sepharose (e.g. Sepharose CL-4B), and composite gels prepared from two kinds of gels such as Superdex 200 combining Dextran (Sephadex^{TM}) and crosslinked Agarose (Superose^{TM}) gels.

In a preferred embodiment the buffer is selected from the group consisting sodium phosphate, ammonium acetate, MES (2-(N-morpholino)ethanesulfonic acid), Bis-Tris (2-bis(2-hydroxyethyl)amino-2-(hydroxymethyl)-1,3-propanediol), ADA (N-(2-Acetamido) iminodiacetic acid), PIPES (piperazine-N,N′-bis(2-ethanesulfonic acid), ACES (N-(2-Acetamido)-2-aminoethanesulfonic acid), BES (N,N-Bis(2-hydroxyethyl)-2-aminoethane-sulfonic acid), MOPS (3-(N-morpholino) propanesulfonic acid), TES (N-Tris(hydroxymethyl)methyl-2-aminoethanesulfonic acid), HEPES (N-2-Hydroxyethyl-piperazine-N′-2-ethanesulfonic acid), preferably sodium phosphate or ammonium acetate, more preferably sodium phosphate.

Optionally said buffer comprises in addition an inorganic salt, preferably a halide of an alkaline metal, more preferably potassium chloride or sodium chloride, most preferably sodium chloride, wherein the concentration of said inorganic salt is about 0 to 500 mM, preferably 0 to 300 mM, most preferably about 0 to 50 mM. In a preferred embodiment the buffer is salt free.

In a particularly preferred embodiment, the product-contacting buffers for the step (b) of SEC (equilibration, load, elution) contain an antioxidant, such as L-methionine. Alternative antioxidants include t-butyl-4-methoxyphenol, 2,6-bis(1,1-dimethylethyl)-4-methyl phenol; potassium or sodium bimetabisulfite, sodium bisulfite.

The size exclusion chromatography further comprises the step of eluting the glycoprotein from said gel filtration matrix by isocratic elution, i.e. the elution buffer has about the same, preferably the same composition as the buffer used for equilibration and/or loading. The flow through may be recorded by UV absorption at 280 nm and the fraction containing the glycoprotein is collected.

Additional Steps

Further to the three main chromatography steps (a), (b) and (c) the process of the present invention may optionally include additional steps known to the person skilled in the art, e.g. chromatography steps, filtration steps or virus inactivation steps. Preferred additional steps are ion exchange chromatography such as anion exchange chromatography or cation exchange chromatography, affinity chromatography such as dye affinity chromatography, immune affinity chromatography, lectin affinity chromatography or hydrophobic interaction chromatography, filtration such as diafiltration, ultrafiltration or nanofiltration, or virus inactivation.

Anion Exchange Chromatography Step (d)

In a preferred embodiment the process of the present invention in addition comprises an anion exchange chromatography (d). The anion exchange chromatography is usually performed by equilibrating and loading the column, followed by a wash and subsequent elution.

The anion exchange chromatography is carried out, preferably with a quaternary ammonium resin, such as CaptoQ (obtainable from GE Healthcare), or a resin having similar characteristics such as ToyoPearl QEA (obtainable from Tosoh), Q Sepharose FF (obtainable from GE Healthcare) or Fractogel EMD, Fractogel TMAE or Fractogel HICAP (obtainable from Merck KGaA, Darmstadt Germany).

The anion exchange chromatography resin is preferably equilibrated, loaded and washed by using a buffer having a mildly alkaline pH, e.g. at or about 6.5 to at or about 9.0, or at or about 7.0 to at or about 8.0, most preferably at or about 7.5. Suitable buffers include, for example borate buffer, triethanolamine/iminodiacetic acid, Tris (2-Amino-2-hydroxymethyl-propane-1,3-diol), sodium phosphate, ammonium acetate, tricine (N-(Tri(hydroxymethyl)methyl)glycine), bicine (2-(bis(2-hydroxyethyl)amino)ethanoic acid), TES, HEPES, TAPS (N-Tris(hydroxymethyl)methyl-3-aminopropanesulfonic acid). Most preferred is sodium phosphate, at a pH of at or about 7.5.

Elution from the ion-exchange resin is achieved by increasing the conductivity of the mobile phase through the addition of salt, preferably NaCl. Suitable buffers include, for example borate buffer, triethanolamine/iminodiacetic acid Tris, ammonium acetate, tricine, bicine, TES, HEPES, TAPS. Preferred is sodium phosphate.

The anion exchange chromatography can be utilized to selectively elute different charge isoforms mainly originating from different sialylation and/or sulfation levels of the glycan-moieties of the glycoprotein.

Glycoproteins are build up from a peptide backbone and oligosaccharides either attached to the OH-group of serine and/or threonine residues in an O-linked fashion and/or attached to the amide group of asparagine in an N-linked fashion. The oligosaccharide structures often terminate with the negatively charged saccharide neuraminic acid (also named sialic acid).

The in vivo activity of glycoprotein products seems to be influenced by the degree of sialylation of terminal galactose. For instance, Erbayraktar et al. (2003, PNAS 100(11), 6741-6746) showed that desialylated EPO (asialoEPO) possesses a very short half-life in the circulation, but nevertheless has neuroprotective activities. Furthermore, due to the short plasma half-life, asialoEPO in contrast to normal recombinant EPO, has no effect on erythropoiesis and thus, does not increased the hematocrit of the patient. Therefore, for specific applications of EPO, different isoforms with different sialylation degrees may be required.

The term “isoform”, as used herein, refers to a glycoprotein preparation/fraction that contains glycoproteins which have identical or very similar amino acid sequence and a common isoelectric point but which may differ in respect to the extent, to the complexity, to the nature, to the antennarity and to the order of attached galactosyl- and sialyl-groups. An isoform according to the invention may also comprise multiple glycoprotein forms of the same or very similar amino acid sequence and isoelectric point which differ additionally in other charge carrying modifications such as acetylation and sulfation. The term “very similar amino acid sequence” indicates that the amino acid sequence of a protein also comprises those sequences that are functionally equivalent to the wild type amino acid sequence and thus, exert the same function. In particular, “very similar amino acid sequence” shares a sequence homology, preferably a sequence identity, with a reference amino acid sequence of at least 70%, preferably at least 80%, at least 90%, at least 95%, most preferably at least 98%, over a stretch of consecutive amino acids representing at least 50%, preferably at least 70%, at least 80%, at least 90%, at least 95%, more preferably 100% of the entire reference amino acid sequence.

Thus, glycoprotein isoforms preferably can be defined by their isoelectric point and amino acid sequence and each such defined isoform may actually comprise multiple isoforms in the strict chemical sense (molecules having the same atomic composition but differing in their spatial structure). In particular, the isoelectric point of different glycoproteins of the same isoform preferably does not differ by more than 2 units, more preferable not more than 1 unit, not more than 0.5 units or not more than 0.2 units, and most preferably the isoelectric point does not differ by more than 0.1 units.

For the selective elution of differently charged isoforms such as differently sialylated isoforms it is preferred to use two or more, preferably two elution buffers A and B which differ in pH and/or salt content, each of them being based on e.g. ammonium acetate, borate buffer, triethanolamine/iminodiacetic acid, Tris, sodium phosphate, ammonium acetate, tricine, bicine, TES, HEPES or TAPS, preferred is sodium phosphate. Using different elution buffers, elution can be performed in a stepwise fashion, first using one elution buffer and then using the other elution buffer, optionally also using one or more intermediate elution steps with different mixtures of the elution buffers. Alternatively or additionally, elution can be performed using a gradient, starting with a first mixing ratio of the elution buffers (e.g. 100% of the first elution buffer) and gradually changing to a second mixing ratio of the elution buffers (e.g. 100% of the second elution buffer).

The elution buffer used first (buffer A) in general can be a) a mildly acidic buffer which is salt-free, or b) a neutral or mildly basic buffer with low salt content such as NaCl (preferably between 20 and 200 mM). Buffer A can be used to elute glycoprotein of low charge, e.g. low degree of sialylation. In variant a) buffer A has a pH e.g. at or about 3.0 to at or about 6.5, or at or about 4.0 to at or about 6.0, most preferably at or about 5. In variant b) buffer A has a pH e.g. at or about 7.0 to 9.0, preferably 8.5.

The elution buffer used second (buffer B) in general is a salt-containing mildly alkaline buffer of a higher salt content than buffer A which can be used to elute glycoprotein of high charge, e.g. high degree of sialylation. Buffer B has a pH e.g. at or about 7.0 to at or about 9.0, or at or about 8.0 to at or about 9.0, most preferably at or about 8.5. The salt is preferably NaCl. The salt content in buffer B is preferably from 200 mM to 1 M.

Using different elution buffers and a gradient or stepwise elution, the different glycoprotein isoforms loaded onto the anion exchange chromatography column will elute in different fractions depending on their charge. For example, the glycoprotein to be purified may be present in the fractions of the flow-through, i.e. it binds to the anion exchange chromatography column only weakly or not at all, it may be eluted with the first elution buffer, at a specific mixing ratio of the first and second elution buffer, or with the second elution buffer. The glycoprotein fractions which are used for the further purification steps and thus, the glycoprotein isoforms which are to be purified, mainly depend on the desired applications of the glycoprotein. The other glycoprotein isoforms which are not of interest can be removed using the anion exchange chromatography step. With respect to EPO, for example only EPO having a high degree of sialylation and thus, having a high circulation half-life, or only EPO having a low degree of sialylation such as asialoEPO and thus, having a low circulation half-life, may be purified.

In a particularly preferred embodiment the product-contacting buffers for the ion-exchange chromatography (equilibration, wash, elution) contain an antioxidant, preferably L-methionine. Alternative antioxidants are mentioned above.

As an alternative or additionally to standard anion exchange chromatography, chromatofocusing can be performed. Chromatofocusing is a chromatography technique that separates proteins according to differences in their isoelectric point (p1). In particular, a charged stationary phase can be used and the proteins loaded onto the chromatofocusing column can be eluted using a pH gradient. For example, the stationary phase may be positively charged and the pH gradient may develop from a first pH to a second, lower pH, for example from about pH 9 to about pH 6 or from about pH 7 to about pH 4. Due to the specific conditions of the chromatofocusing, proteins elute in order of their isoelectric points and preferably proteins of a specific pI are focused into narrow bands. This, as proteins at a pH higher than their pI are negatively charged and attach to the positively charged stationary phase, thereby being slowed down. When the pH in the elution gradient reaches the pI of the protein, it is overall neutral in charge and thus migrates with the flow of the mobile phase. At a pH lower than the pI of the protein, the protein is repulsed by the stationary phase due to its positive charge, thus accelerating it. Thereby proteins at the rear of a zone will migrate more rapidly than those at the front, gradually forming narrower bands of proteins. In this setting, the protein with the highest pI elutes first and the protein with the lowest pI will elute last.

Suitable stationary phases are, for example, media substituted with charged, buffering amines such as Mono P (obtainable from GE Healthcare) or other anion exchange chromatography material. For forming the pH gradient for elution, suitable buffing systems such as Polybuffer 74 or Polybuffer 96 (obtainable from GE Healthcare) can be used. Equilibration, loading and washing of the column can be done using any condition where the glycoprotein of interest and/or any impurities bind to the column material. For example, conditions as described above for the anion exchange chromatography can be used. When using a decreasing pH gradient, preferably a buffer having a pH equal to or higher than the starting pH of the elution gradient is used for equilibration, loading and/or washing. When using an increasing pH gradient, preferably a buffer having a pH equal to or lower than the starting pH of the elution gradient is used for equilibration, loading and/or washing. Preferably, for equilibration, loading and washing, a buffer similar to that used at the beginning of the elution pH gradient is used.

Overall Process

The steps of reverse phase chromatography, boronate affinity chromatography, and size exclusion chromatography may be carried out in any order, although it is preferred to carry out a step of reverse phase chromatography first. The remaining steps may be carried out in any order, although it is preferred to follow the order of (1) reverse phase chromatography, (2) boronate affinity chromatography, (3) size exclusion chromatography, and optionally anion exchange chromatography (4). Optional is a subsequent concentration and/or buffer exchange step (5) of ultrafiltration and/or diafiltration, and a step (6) of nanofiltration.

In preferred embodiments, the process for the purification of a glycoprotein according to the invention does not comprise an immunoaffinity chromatography and/or a cation exchange chromatography. More preferably, the process according to the invention does not comprise any further chromatographic steps except of those described herein. The process according to the invention preferably comprises only three chromatographic steps, i.e. a reverse phase chromatography, a boronate affinity chromatography and a size exclusion chromatography, or only four steps, i.e. a reverse phase chromatography, a boronate affinity chromatography, a size exclusion chromatography, and an anion exchange chromatography. The anion exchange chromatography may also be replaced by a chomatofocusing step as described above.

However, further non-chromatographic steps, preferably those described herein, may be performed in addition to and also between the steps defined. Preferably, these further steps include steps for diminishing or inactivation undesired or hazardous substances such as bacteria, viruses, nucleic acids or prion proteins, for example sterile filtration, nanofiltration, adsorption and/or pH inactivation steps. In alternative embodiments, besides the steps described above, the process according to the invention may comprise chromatographic steps for diminishing or inactivation undesired or hazardous substances, including for example adsorption chromatography. Preferably, the purification process of the invention comprises at least one, more preferably at least two, most preferably at least three virus diminishing or inactivation steps. In this respect, also the chromatography steps of the purification process according to the invention, in particular the size exclusion chromatography step (c), may be used as virus diminishing step since they normally separate viruses from the glycoprotein. For example, viruses and virus-like particles have a much bigger size compared to glycoproteins and thus, are effectively separated therefrom during size exclusion chromatography.

Furthermore, the process according to the invention preferably does not comprise a buffer exchange step between the different chromatography steps. In particular, if the process is performed in the order of (1) reverse phase chromatography, (2) boronate affinity chromatography, and (3) size exclusion chromatography, preferably there is no buffer exchange between the reverse phase chromatography and the boronate affinity chromatography and/or between the boronate affinity chromatography and the size exclusion chromatography. Likewise, the process according to the invention preferably does not comprise a concentration step between the different chromatography steps. In particular, if the process is performed in the order of (1) reverse phase chromatography, (2) boronate affinity chromatography, and (3) size exclusion chromatography, preferably there is no concentration between the reverse phase chromatography and the boronate affinity chromatography and/or between the boronate affinity chromatography and the size exclusion chromatography. Furthermore, in case the optional anion exchange chromatography (d) is performed after the size exclusion chromatography (c), preferably there is no buffer exchange step and/or concentration step between these two chromatographic steps.

According to the invention, a buffer exchange step includes the removal of one or more components form the glycoprotein solution and optionally the addition of one or more other components to the glycoprotein solution. Buffer exchange steps are for example diafiltration or dialysis steps. In particular, the addition of a further component to the glycoprotein solution alone is not a buffer exchange. According to the invention, a concentration step is a step wherein the concentration of the glycoprotein of interest is increased. Particular concentration steps are ultrafiltration steps, concentration-increasing dialysis steps, solvent evaporation steps or precipitation steps.

### Other Steps

Prior to the first chromatography step (particularly prior to a step of reverse phase chromatography), it may be desirable to carry out a step of ultrafiltration, in order to concentrate the crude glycoprotein. Furthermore, additionally a step of diafiltration may be performed prior to the first chromatography step in order to perform a buffer exchange. The ultrafiltration step and the diafiltration step may be performed simultaneously or sequentially. The ultrafiltration and/or diafiltration is preferably carried out using a membrane having a cut-off of at or about 3-20 kD, most preferably at or about 10 kD. However, the present invention also encompasses purification processes wherein no ultrafiltration step and/or no diafiltration step is performed prior to the first chromatography step.

In a preferred embodiment, after one or more of the steps of chromatography (particularly after the last step of chromatography), the glycoprotein sample is subjected to an ultrafiltration and/or diafiltration step. Preferably the ultrafiltration and/or diafiltration is performed in order to obtain a bulk having the desired composition. The ultrafiltration (and/or diafiltration) is preferably carried out using a membrane having a cut-off of at or about 3-30 kD, most preferably at or about 10 kD. It is preferred to perform during ultrafiltration and/or diafiltration a buffer exchange to a pre-formulation buffer, e.g. selected from the group consisting of sodium phosphate, sodium citrate, MES, Bis-Tris, ADA, PIPES, ACES, BES, MOPS, TES, HEPES, preferably sodium phosphate, preferably sodium-phosphate containing stabilizers e.g. sucrose and antioxidants like L-methionine. The pH preferably is in the range of 6.5 to 7.5, more preferably about 7.0 to 7.1. Preferably, an ultrafiltration step is performed prior to the chromatography steps, and/or prior to the size exclusion chromatography, and/or after all chromatography steps.

Further optional steps which can be performed in the purification process according to the invention include one or more sterile filtration steps. These steps can be used to remove biological contaminations such as eukaryotic and/or prokaryotic cells, in particular bacteria, and/or viruses. Preferably, these steps are preformed at or near the end of the purification process to prevent a further contamination after the sterile filtration step. For removal of bacteria or other cells, the filter used for sterile filtration preferably has a pore size of 0.22 μm or less, preferably 0.1 μm or less. For removal of viruses or virus-like particles, a nanofiltration step as described below may be performed. Furthermore, one or more filtration steps for clarifying the glycoprotein solution can be performed. Preferably, a 2 μm depth filter is used for clarification. In particular embodiments, a filtration step for clarifying the glycoprotein solution is performed prior to the chromatography steps and/or after the reverse phase chromatography.

Another additional step which can be performed in the purification process according to the invention is a virus inactivation step via incubation of the glycoprotein at a specific pH. For example, the glycoprotein is incubated at a pH of 4.0 or less, preferably at about pH 3.6. The incubation time preferably is at least 15 min, at least 30 min, at least 60 min, at least 90 min, at least 2 h, at least 3 h or at least 6 h. Incubation may be performed at low temperature such as 10° C. or less or 4° C. or less, or at about room temperature. For example, the glycoprotein material may be incubated at a pH of about 3.6 for about 90 min at about room temperature. This virus inactivation step can be performed at any time during the purification process and preferably is performed after the last chromatography step.

In one preferred embodiment, the process of the present invention comprises the following steps in the order shown below:

(0) Ultrafiltration (optionally an additional diafiltration step; preferably with a membrane having a cut-off of at or about 10 kD);

(1) Reverse phase chromatography (RPC) (preferably using a Source 30 RPC column);

(2) Boronate affinity chromatography (preferably using a ProSep-PB column);

(3) Size exclusion chromatography (preferably using a Superdex 200 column);

(4) Anion-exchange chromatography (preferably using a CaptoQ column);

(5) Ultrafiltration and/or diafiltration (preferably with a membrane having a cut-off of 10 kD).

It may be desirable to subject the glycoprotein sample to a step of nanofiltration, in particular as a virus clearance step; i.e. to reduce the risk of contamination of the glycoprotein preparation with viruses or virus-like particles originating from the cell culture. Nanofiltration may be performed at any stage of the purification process, however, it is particularly preferred to carry out nanofiltration after the end of the chromatographic procedure. Nanofiltration may be performed more than one time, for example it may be performed twice. Preferred nanofiltration devices have a pore size of about 15 to 20 nm.

In another preferred embodiment, the method of the invention thus comprises the following steps in the order shown below:

(0) Ultrafiltration (preferably with a membrane having a cut-off of at or about 10 kD);

(1) Reverse phase chromatography (RPC) (preferably using a Source 30 RPC column);

(2) Boronate affinity chromatography (preferably using a ProSep-PB column);

(3) Ultrafiltration (preferably with a membrane having a cut-off of at or about 10 kD);

(4) Size exclusion chromatography (preferably using a Superdex 200 column);

(5) Anion-exchange chromatography (preferably using a CaptoQ column);

(6) Ultrafiltration and/or diafiltration (preferably with a membrane having a cut-off of 10 kD);

(7) Nanofiltration (preferably including virus clarification).

The specific purification processes described above are preferably performed without including any further chromatography steps and/or ultrafiltration steps and/or diafiltration steps. However, in particular embodiments, the purification processes described above may further comprise additional steps, in particular one or more of the additional steps described herein, for example those used for removing or inactivating undesired or and/or hazardous substances.

It is preferred that an antioxidant or a free amino acid or dipeptide with antioxidant and scavenging effect is included in some or all of the steps of the purification method according to the present invention. More specifically the antioxidant is present in any of the buffers used to purify and/or concentrate and/or filter the glycoprotein such as EPO. The antioxidant prevents oxidation of the glycoprotein such as EPO during processing. A preferred antioxidant is L-methionine. Preferably, L-methionine is used at a concentration of at or about 0.1 to 10 mM. Further examples of an antioxidant include t-butyl-4-methoxy-phenol, 2,6-bis(1,1-dimethylethyl)-4-methyl phenol; potassium or sodium bimeta-bisulfite, sodium bisulfite. Examples of free amino acid and dipeptide with antioxidant and scavenging effect are histidine, taurine, glycine, alanine, carnosine, anserine, 1-methylhistidine or combinations thereof.

An advantage of the present invention is that the purification process is highly effective, reduces the number chromatographic steps to a minimum of 3 chromatographic steps or—including an enrichment of highly or sparsely sialylated glycoprotein molecules—to a minimum of 4 chromatographic steps. In particular, using the purification process according to the invention, cost intensive and problematic purification steps such as in particular affinity purification steps, especially immunoaffinity purification steps, become unnecessary and can be avoided. Furthermore, using the purification process according to the invention potentially yield-limiting denaturing conditions during the purification such as the use of urea-containing elution buffers can be avoided. The process provides a high degree of glycoprotein purity and specific bioactivity >90%, preferably >98%, more preferably >99% w/w, each based on total protein as measured, for example, by HCP-ELISA. Furthermore, the purification process according to the invention provides a surprisingly high recovery of the glycoprotein of interest present in the starting material.

The Glycoproteins

Glycoproteins are proteins that contain oligosaccharide chains (glycans) covalently attached to polypeptide side-chains. Glycoproteins may comprise one or more glycans which preferably are coupled to a nitrogen atom (N-glycosylation) or an oxygen atom (O-glycosylation) of the polypeptide. Thus, the glycoprotein may be N-glycosylated and/or O-glycosylated. Preferably, the glycoproteins comprise natural glycans. However, the term “glycoprotein” comprises proteins or polypeptides having natural glycans and/or non-natural glycans, in particular synthetically produced glycans and/or glycans comprising non-natural or modified monosaccharide unit(s).

The glycoprotein to be purified is preferably selected from the group of cytokine glycoproteins and/or glycoprotein hormones, in particular glycosylated interferones such as INF-β and INF-γ, glycosylated interleukins such as IL-2, colony stimulating factors such as M-CSF, GM-CSF and G-CSF, thrombopoietin (TPO) and in particular erythropoietin (EPO). However, the process for purification of a glycoprotein according to the invention is also suitable for purifying other glycoproteins such as, for example, gonadotropins such as FSH (follicle-stimulating hormone), CG (chorionic gonadotropin), LH (luteinizing hormone) and TSH (thyroid-stimulating hormone), various antibodies, in particular monoclonal antibodies, and tissue plasminogen activator.

According to the invention, the term “glycoprotein” also includes any isoforms, fragments, fusion proteins, variants and analogues of the glycoproteins specifically mentioned herein, especially those described below and under step (d) (AEX) above. Preferably, the isoforms, fragments, fusion proteins, variants and analogues of glycoproteins exhibit one or more biological activities of the natural glycoprotein from which they are derived. The glycoproteins may refer to the naturally occurring glycoproteins as well as to recombinant versions thereof.

### Storage/Lyophilisation

The liquid composition resulting from the purification process as described above and containing purified glycoprotein may be frozen for storage as is, or after purification, the eluate may be subjected to lyophilisation (“freeze-drying”) to remove solvent. The resulting liquid or lyophilised product is termed “Glycoprotein Bulk”.

Formulations

The glycoprotein of the invention or purified according to the method of the invention may be formulated for any kind of administration, preferably for injection, either intramuscular or subcutaneous, preferably subcutaneous. The glycoprotein formulation may be freeze-dried, in which case it is dissolved in water for injection just prior to injection. The glycoprotein formulation may also be a liquid formulation, in which case it can be injected directly, without prior dissolution. The formulation may contain known excipients and stabilizers and may additionally comprise antioxidants and/or surfactants. The glycoprotein formulation may be single dose or multiple dose. If it is multiple dose, it should preferably contain a bacteriostatic agent, such as, for example, alkylparabene, benzyl alcohol, meta-cresol, thymol or phenol, preferably methylparabene or meta-cresol. Single dose formulations may also comprise a bacteriostatic agent.

Indications

The glycoprotein of the invention is suitable for use in all treatments where the glycoprotein is indicated.

Recombinant Glycoproteins

The use of the term “recombinant” refers to preparations of glycoprotein such as EPO that are produced through the use of recombinant DNA technology. One example of a method of expressing a glycoprotein using recombinant technology is the transfection of a suitable host cell, preferably a eukaryotic host cell, with an expression vector comprising a DNA sequence encoding the glycoprotein of interest. Usually, the expression vector carries a strong promoter driving the expression of the glycoprotein, e.g. CMV or SV40 and a suitable selection marker for selecting host cells that have incorporated the vector. Transfection can be stable or transient. Suitable recombinant expression systems are well-known in the prior art and thus need no detailed description. Preferably, the eukaryotic host cell is selected from primate cells, preferably human cells and rodent cells, preferably CHO cells. For recombinant expression of EPO, the eukaryotic host cells are transfected with DNA sequences encoding the EPO protein sequence, provided on a vector harbouring a suitable promoter, as is common practice in the art. The DNA encoding EPO may be a cDNA or it may contain introns.

Another example of the use of recombinant technology to produce EPO is by the use of homologous recombination to insert a heterologous regulatory segment in operative connection to endogenous sequences encoding EPO, as described in European patent no. EP 0 505 500 (Applied Research Systems ARS Holding NV).

The purification process according to the invention is useful for purifying natural as well as recombinant glycoproteins, including isoforms and variants thereof. Glycoprotein isoforms preferably refer to isoforms as defined above. The term “variant” preferably encompasses glycoproteins derived from a natural glycoprotein, such as mutant forms thereof, fusion proteins thereof, fragments thereof and/or glycoproteins having a different glycosylation pattern. Also mimetic compounds of the glycoproteins are comprised, including proteins comprising glycomimetic structures and/or peptidomimetic structures. Preferably, the glycoprotein variants and/or isoforms exhibit one or more activities which are qualitatively and/or quantitatively similar or identical to those of the natural glycoprotein.

The expression “glycoprotein variant” such as “EPO variant” is meant to encompass those molecules differing in amino acid sequence, number of glycosylation sites (including additional or deleted glycosylation sites) and/or glycosylation pattern from human glycoprotein but exhibiting one or more of its activities. Examples of EPO variants include epoetins which comprise the same amino acid sequence as the natural erythropoietin but have a different glycosylation pattern, such as epoetin alpha (epoetin α), epoetin beta (epoetin β), epoetin gamma (epoetin γ), epoetin delta (epoetin δ), epoetin epsilon (epoetin ε), epoetin zeta (epoetin ζ), epoetin theta (epoetin θ), epoetin kappa (epoetin κ) and epoetin omega (epoetin ω). Further examples of EPO variants are:
darbepoetin α, comprising additional glycosylation sites due to the exchange of 5 amino acids;
fusion proteins of EPO such as a fusion protein of EPO and albumin (e.g. albupoetin), an EPO dimer fusion protein, a fusion protein of EPO and the Fc region of an antibody, a fusion protein of EPO and human chorionic gonadotropin (hCG); or a fusion protein of EPO and GM-CSF;
CEPO (carbamylated EPO), wherein a carbamyl group is coupled to most, preferably all lysin residue of EPO;
asialoEPO, an EPO variant which comprises only a few sialic acid residues such as 5 or less, 4 or less, 3 or less, 2 or less or only 1 sialic acid residues, or no sialic acid residues at all.

The EPO proteins and variants referred to herein also include EPO from different species like e.g. horse (Equus caballus), pig (Sus scrofa), cow (Bos taurus), cat (Felis catus), and dog (Canis familiaris).

In a preferred embodiment, the EPO is produced recombinantly, either in a serum or in a serum-free medium. In another preferred embodiment, the purified EPO produced according to the method of the invention is suitable for subcutaneous administration, permitting self-administration by the patient.

The variants of the glycoprotein described above with respect to EPO as exemplary glycoprotein in a similar manner also apply to other glycoproteins, where appropriate, in particular to those glycoproteins mentioned above.

The expression “crude recombinant glycoprotein” refers to the cell culture supernatant from recombinant cells expressing glycoprotein, before it has undergone any chromatographic step. The expression encompasses the raw form of the supernatant (as isolated from cells) as well as concentrated and/or filtered and/or ultrafiltered supernatant.

Process for Manufacturing Glycoproteins

Also provided is a process for manufacturing a glycoprotein of interest by performing the process for the purification of a glycoprotein described herein. The glycoprotein can be obtained from natural sources or recombinantly.

In a preferred embodiment, a process for manufacturing a glycoprotein of interest is provided, comprising the following steps:
i) recombinantly expressing the glycoprotein of interest;
ii) purifying said recombinantly expressed glycoprotein of interest by subjecting a liquid containing said glycoprotein at least to the steps of:
   a) reverse phase chromatography,
   b) boronate affinity chromatography, and
   c) size exclusion chromatography.

The respective manufacturing process leads to the production of very pure glycoproteins which are in particular suitable for use in pharmaceutical formulations.

Said manufacturing process preferably comprises at least one or more steps as described above in conjunction with the purification process. The respective disclosure also applies to the manufacturing process according to the present invention and it is referred to the above disclosure to avoid repetitions.

Furthermore, the manufacturing process according to the present invention may comprise a step of formulating the glycoprotein of interest in form of a pharmaceutical formulation. Suitable liquid or lyophilised formulations are known in the prior art and are described above, we refer to the respective disclosure.

Preferably, the glycoprotein of interest that is produced by the manufacturing method according to the present invention is selected from the group of cytokine glycoproteins, preferably EPO.

Experimental Section

The following experiment illustrates the process of the present invention and in no way is intended to limit the disclosure.

Step 0: Filtration

The crude EPO forming the starting material was derived from cell culture supernatants containing recombinant EPO.

Prior to the reverse phase chromatography step the supernatant was clarified by room temperature filtration through a 2 μm depth filter.

Step 1: Reverse Phase Chromatography (Source30 RPC Column)

Loading buffer: dd H₂O/10% v/v isopropanol (containing 0.1% TFA)

Elution buffer: dd H₂O/60% v/v isopropanol (containing 0.1% TFA)

The material obtained from the filtration step (0) was supplemented with isopropanol at a concentration equivalent to the loading buffer. The Source 30 column is equilibrated with loading buffer. After loading the material onto the column unbound material is washed out for about 15 CV by loading buffer. The EPO is eluted by increasing the isopropanol concentration up to 60% v/v in a linear fashion over 8 CV. The step is performed at room temperature.

RPC

| Column | Source 30 RPC |
|---|---|
| | Polystyrene/divinyl benzene |
| Bonded phase | None |
| Bead form | Rigid, spherical, porous, |
| | monodisperse |
| Particle size | 30 μm |
| Residence [min] | 1.3 |
| max lin Flow [cm/h] | 480 |

Step 1a: Dilution and Filtration

The eluate from step (1) (RPC) was diluted at room temperature 1:5 with the borate loading buffer for the next step. Prior to the affinity chromatography step the supernatant was clarified by room temperature filtration through a 2 μm depth filter.

Step 2: Affinity Chromatography (ProSep PB)

Loading buffer: 0.1M Borate+0.15M NaCl pH 8.5

Elution buffer: 200 mM Tris pH 7.6

The ProSepPB column is equilibrated with loading buffer. After loading the material onto the column unbound material is washed out for about 8 CV by loading buffer. The EPO is eluted by one step rise to 100% of elution buffer (about 5 CV). The step is performed at room temperature.

Step 2a: Ultrafiltration

The eluate from step (2) (AC) was subjected at room temperature to ultrafiltration with a membrane having a cut-off at or about 10 kD at a transmembrane pressure not exceeding 1.2 bar and concentrating the eluate to about 10% of the SEC column volume.

Step 3: Size Exclusion Chromatography (Superdex 200 Column)

Running buffer: 20 mM Na-phosphate pH 7.5

The pooled material from step (2a) was subjected to the SEC column, equilibrated with running buffer. EPO is eluted under isocratic conditions at a distinct retention time (about 0.6-0.7 CV). This chromatography step provides purification and a buffer exchange prior to the next step. The SEC step is performed at room temperature.

SEC

| | |
|---|---|
| Column | Superdex 200 |
| | Spherical composite of cross-linked |
| | agarose and dextran |
| Bed height | 60 cm |
| Exclusion limit | 1.3 × 10⁶ globular protein |
| (Mᵣ) | |
| Separation range | 10 000-600 000 globular protein |
| (Mᵣ) | |
| max lin Flow | 120 |
| [cm/h] | |

Step 4: Anion-Exchange Chromatography (CaptoQ Column)

Loading buffer: 20 mM Na-phosphate, pH 7.5

Elution buffer: 20 mM Na-phosphate, 0.5M NaCl, pH 7.5

The material obtained from step (3) (SEC) was then applied to an anion exchange resin equilibrated with loading buffer. The unbound material was washed out with loading buffer (about 10 CV). EPO was partly eluted by 10% of elution buffer (containing the less charged EPO molecules) prior to the second elution step with 50% of elution buffer (containing the higher charged EPO molecules) and a last elution step with 100% Elution buffer to solve all bound components from the column. All three elution steps are performed in a stepwise fashion. The AEX is performed at room temperature.

AEX

| | |
|---|---|
| Matrix | CaptoQ |
| Ion exchange type | strong anion, Q |
| Charged group | —N⁺(CH₃)₃ |
| Total ionic capacity | 0.16-0.22 mmol Cl⁻/ml |
| | medium |
| Particle size* | 90 μm (d50v) |
| Max. Lin Flow | 700 cm/h |
| Dynamic binding capacity | >100 mg BSA/ml medium |

Step 5: Diafiltration (Membrane Having a Cut-Off of 10 kD)

Diafiltration against a desired formulation buffer.

The eluate from step (4) (AEC) was then applied at room temperature to diafiltration. By this step buffer is exchanged to preformulation buffer and adjusted to the desired concentration.

Step 6: Nanofiltration

The product from the diafiltration step was directly applied to a 20 nm nanofiltration device at a pressure of about 2 bar. The step was performed at room temperature.

The process of steps (0) to (6) rendered EPO at a purity of >99.99% w/w based on total protein as determined by HCP-Assay (host cell protein level <0.01% w/w).

1. A process for purification of a glycoprotein comprising subjecting a liquid containing the glycoprotein to the steps of:
a) reverse phase chromatography,
b) boronate affinity chromatography, and
c) size exclusion chromatography.

2. The process according to claim 1, wherein the reverse phase chromatography, boronate affinity chromatography, and size exclusion chromatography steps are performed sequentially.

3. The process according to claim 1, wherein the reverse phase chromatography step comprises the use of an elution buffer containing an organic solvent.

4. The process according to claim 1, wherein the boronate affinity chromatography step comprises elution under non-denaturing conditions.

5. The process according to claim 1, further comprising one or more steps selected from the group consisting of chromatography, filtration and virus inactivation.

6. The process according to claim 1, further comprising one or more steps selected from the group consisting of ion exchange chromatography, affinity chromatography, diafiltration, ultrafiltration, nanofiltration and virus inactivation.

7. The process according to claim 1, wherein buffer exchange is not performed between the reverse phase chromatography and boronate affinity chromatography steps.

8. The process according to claim 1, further comprising the step of subjecting the liquid containing the glycoprotein to anion exchange chromatography.

9. The process according to claim 8, wherein the anion exchange chromatography step is carried out subsequent to the size exclusion chromatography step.

10. The process according to claim 8, wherein different charged isoforms of the glycoprotein are separated.

11. The process according to claim 1, wherein the glycoprotein is a cytokine glycoproteins.

12. The process according to claim 1, wherein the glycoprotein is produced recombinantly.

13. The process according to claim 1, comprising sequentially subjecting the liquid containing the glycoprotein to the steps of:
(0) Ultrafiltration;
(1) Reverse phase chromatography;
(2) Boronate affinity chromatography;
(3) Ultrafiltration;
(4) Size exclusion chromatography;
(5) Anion-exchange chromatography;
(6) Ultrafiltration and/or diafiltration; and
(7) Nanofiltration.

14. A process for manufacturing a glycoprotein of interest, comprising:
i) recombinantly expressing the glycoprotein of interest;
ii) purifying the recombinantly expressed glycoprotein of interest by subjecting a liquid containing the glycoprotein at least to:
a) reverse phase chromatography,
b) boronate affinity chromatography, and
c) size exclusion chromatography.

15. The manufacturing process according to claim 14, further comprising:
one or more steps selected from the group consisting of ultrafiltration, anion-exchange chromatography, diafiltration, and nanofiltration and/or formulating the glycoprotein of interest in a pharmaceutical formulation.

16. The manufacturing process according to claim 14, wherein the glycoprotein is a cytokine glycoproteins.

17. The method of claim 3, wherein the organic solvent is isopropanol.

18. The method of claim 11, wherein the cytokine glycoprotein is erythropoietin (EPO).

19. The manufacturing process of claim 16, wherein the cytokine glycoprotein is erythropoietin (EPO).
